(19) United States
(12) Patent Application Publication
(10) Pub. No.: US 2026/0318907 A1
(43) Pub. Date: Oct. 15, 2026

(54) BIOCHEMICAL SENSING ARRAYS, METHODS FOR PREPARING THE SAME, TEST STRIPS, AND TETS KITS

(71) Applicant: TAIYUAN UNIVERSITY OF TECHNOLOGY, Jinzhong (CN)

(72) Inventors: Shengbo SANG, Jinzhong (CN); Xingbo YU, Jinzhong (CN); Dong ZHAO, Jinzhong (CN); Haitao ZHANG, Jinzhong (CN); Xing GUO, Jinzhong (CN)

(73) Assignee: TAIYUAN UNIVERSITY OF TECHNOLOGY, Jinzhong (CN)

(21) Appl. No.: 19/266,233

(22) Filed: Jul. 11, 2025

(30) Foreign Application Priority Data
Apr. 11, 2025 (CN) 202510454337.8

(51) Int. Cl. G01N 33/543; B01L 3/00

(52) U.S. Cl. CPC G01N 33/54387; B01L 3/5023; B01L 2200/12; B01L 2300/0636; B01L 2300/0825; B01L 2300/123

(57) ABSTRACT

Provided is a biochemical sensing array, a method for preparing the same, a test strip, and a test kit, belonging to the field of biosensor technology. The method includes: providing a substrate; forming a flexible layer on a second surface of the substrate by a microelectronic printing technique; forming a conductive layer on a first surface of the substrate by the microelectronic printing technique; forming a sensitive layer on a side of the flexible layer away from the substrate. The flexible layer and the conductive layer are prepared by the microelectronic printing technique, enabling precise control over a thickness of each of the flexible layer and the conductive layer. This approach enhances the uniformity and reproducibility of biochemical sensors. The prepared biochemical sensing array includes a plurality of biochemical sensors, facilitating high-throughput detection that significantly improves testing efficiency compared to a single biochemical sensor.

### CROSS-REFERENCE TO RELATED APPLICATIONS

This application claims priority to Chinese Patent Application No. 202510454337.8 filed on Apr. 11, 2025, the entire contents of which are hereby incorporated by reference.

### TECHNICAL FIELD

The present disclosure relates to the field of biosensor technologies, and more particularly, to biochemical sensing arrays, methods for preparing the same, test strips, and test kits.

### BACKGROUND

Surface stress biosensors, as a type of biosensor, can be applied in fields such as clinical medical diagnosis, biochemical detection, and new drug discovery. The detection principle of the surface stress biosensors involves modifying the biosensors with probes to achieve qualitative analysis of to-be-tested biomolecules. When a to-be-tested biomolecule binds to the corresponding probe, a sensitive component of the biosensor undergoes a slight deformation. Different concentrations of to-be-tested biomolecules generate varying degrees of stress upon binding to the corresponding probe. By detecting electrical signals corresponding to the different deformations, quantitative analysis of the concentration of the to-be-tested biomolecules can be achieved.

However, in the above-mentioned technologies, film layers of the surface stress biosensors are typically prepared by spin coating, which makes it difficult to precisely control a thickness of each of the film layers. As a result, the uniformity and reproducibility of the prepared biosensors are relatively poor.

Therefore, it is desirable to provide a biochemical sensing array, a preparation method thereof, a test strip, and a test kit. By introducing a microelectronic printing technique to replace the conventional spin coating technique, it can achieve nanoscale precision control over the thickness of each film layer of the sensor, thereby improving the uniformity and detection efficiency of the sensor and overcoming the technical limitations of traditional spin coating in film thickness control.

### SUMMARY

One or more embodiments of the present disclosure provide a method for preparing a biochemical sensing array. The method includes: providing a substrate; the substrate including a first surface and a second surface disposed opposite to each other, the first surface being provided with through-holes arranged in an array, a first trace, and a plurality of second traces; each of the second trace corresponds to one of the through-holes, a first contact point and a second contact point are disposed around a periphery of each of the through-holes, the first trace is electrically connected to all first contact points, and each of the plurality of second traces is electrically connected to the second contact point disposed around the periphery of the through-hole corresponding to the second trace; forming a flexible layer on the second surface by microelectronic printing, wherein the flexible layer at least covers the through-holes; the flexible layer covering at least the through holes; forming a conductive layer on the first surface by microelectronic printing, wherein the conductive layer includes a plurality of separated conductive blocks, each conductive block covering a group of a through-hole and the first contact point and the second contact point disposed around the periphery of the through-hole; and forming a sensitive layer on a side of the flexible layer away from the substrate, wherein the sensitive layer includes a plurality of separated thin-film blocks, each of the thin-film blocks at least partially overlapping with one of the through-holes.

One or more embodiments of the present disclosure provide a biochemical sensing array, the biochemical sensing array being prepared by the method of preparing the biochemical sensing array as described in any of the embodiments of the present disclosure.

One or more embodiments of the present disclosure provide a test strip including the biochemical sensing array as described in any embodiment of the present disclosure.

One or more embodiments of the present disclosure provide a test kit including the biochemical sensing array as described in any of the embodiments of the present disclosure.

The advantageous effects of the embodiments of the present disclosure include, but are not limited to, the following:

According to the embodiments of the present disclosure, the flexible layer and conductive layer are prepared using the microelectronic printing technique. By utilizing printing parameters that can be precisely adjusted, the film thickness of the flexible layer and conductive layer can be accurately controlled, which facilitates improved uniformity and reproducibility of the biochemical sensor. The prepared biochemical sensing array comprises a plurality of biochemical sensors, enabling simultaneous batch detection, and significantly improving detection efficiency compared to a single biochemical sensor.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, where like reference numerals represent similar structures throughout the several views of the drawings, and wherein:

FIG. 1 is a flowchart illustrating an exemplary process of a method for preparing a biochemical sensing array according to some embodiments of the present disclosure;

FIG. 2 is a schematic diagram illustrating an exemplary structure of a first surface of a substrate according to some embodiments of the present disclosure;

FIG. 3 is a schematic diagram showing an exemplary structure of a second surface of a substrate according to some embodiments of the present disclosure.

FIG. 4 is an exemplary schematic diagram illustrating a structure of a flexible layer according to some embodiments of the present disclosure;

FIG. 5 is a schematic diagram illustrating an exemplary structure of a conductive layer according to some embodiments of the present disclosure;

FIG. 6 is a schematic diagram illustrating an exemplary structure of a sensitive layer according to some embodiments of the present disclosure;

FIG. 7 is a schematic diagram illustrating an exemplary structure of a substrate according to some embodiments of the present disclosure; and

FIG. 8 is an exemplary schematic diagram illustrating a detection result of a biochemical sensing array according to some embodiments of the present disclosure.

### DETAILED DESCRIPTION

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown but is to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. The term “and/or” as used in the claims and the specification includes any and all combinations of one or more of the associated listed items. It will be further understood that the terms “comprise,” “comprises,” and/or “comprising,” “include,” “includes,” and/or “including” when used in this disclosure, specify the presence of stated features, integers, steps, operations, elements, and/or components, but do not preclude the presence or addition of one or more other features, integers, steps, operations, elements, components, and/or groups thereof.

It will be understood that the terms “system,” “engine,” “unit,” “module,” and/or “block” used herein are one method to distinguish different components, elements, parts, sections, or assemblies of different levels in ascending order. However, the terms may be displaced by another expression if they achieve the same purpose.

It will be understood that, although the terms “first,” “second,” “third,” etc., may be used herein to describe various elements, these elements should not be limited by these terms. These terms are only used to distinguish one element from another. For example, a first element could be termed a second element, and, similarly, a second element could be termed a first element, without departing from the scope of exemplary embodiments of the present disclosure.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The flowcharts used in the present disclosure illustrate operations that systems implement according to some embodiments in the present disclosure. It is to be expressly understood, the operations of the flowchart may be implemented not in order. Conversely, the operations may be implemented in an inverted order, or simultaneously. Moreover, one or more other operations may be added to the flowcharts. One or more operations may be removed from the flowcharts.

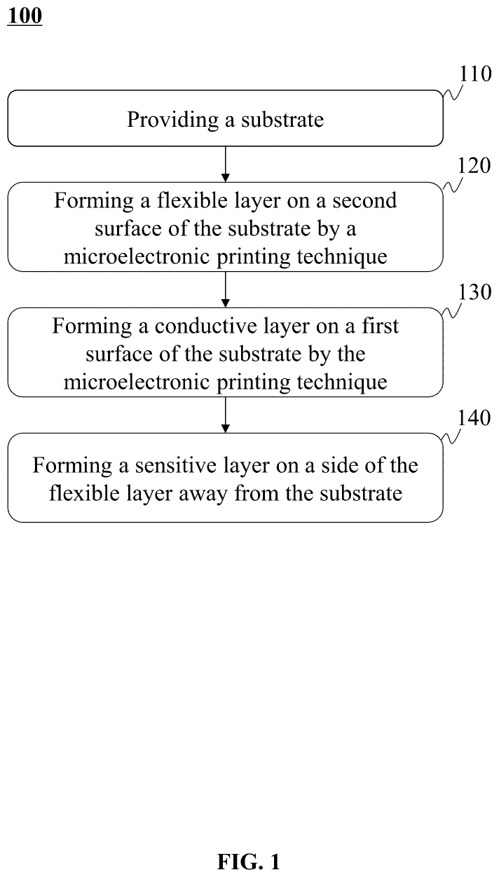

FIG. 1 is a flowchart of an exemplary process of a method for preparing a biochemical sensing array according to some embodiments of the present disclosure.

As shown in FIG. 1, process 100 includes the following operations. In some embodiments, process 100 may be implemented by an operator or a preparation device. The preparation device may include a processor. The preparation device may further include a heating device, a printing device, a magnetron sputtering device, or the like.

In 110, providing a substrate.

The biochemical sensing array refers to a miniaturized detection system integrating a plurality of independent sensing units (e.g., biochemical sensors). The biochemical sensing array is capable of analyzing various biomolecules or chemical substances simultaneously or in parallel. The core principle of the biochemical sensing array includes converting a biochemical signal into a measurable physical signal (e.g., an electrical signal) through an interaction between a specific recognition element (e.g., an enzyme, an antibody, a DNA probe, etc.) and a target object, followed by data processing to achieve high-throughput multi-component detection.

The substrate refers to a foundational layer of the biochemical sensing array, used to support other functional layers.

In some embodiments, the substrate 10 includes a flexible substrate, such as a flexible printed circuit (FPC).

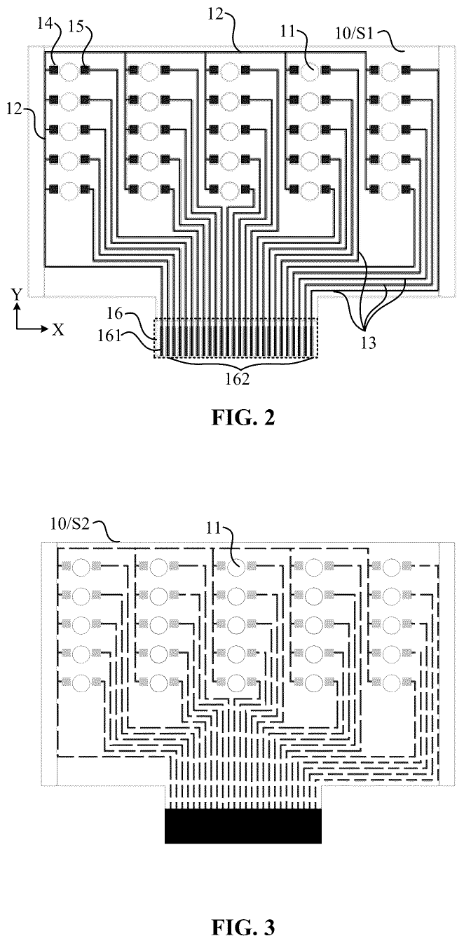

FIG. 2 is a schematic diagram illustrating an exemplary structure of a first surface of a substrate according to some embodiments of the present disclosure. FIG. 3 is a schematic diagram showing an exemplary structure of a second surface of a substrate according to some embodiments of the present disclosure.

In some embodiments, as shown in FIGS. 2 to 3, the substrate (e.g., a substrate 10) includes a first surface S1 and a second surface S2 disposed opposite to each other. The first surface S1 is provided with through-holes 11 arranged in an array, a first trace 12, and a plurality of second traces 13. Each of the plurality of second traces 13 corresponds to one of the through-holes 11. A first contact point 14 and a second contact point 15 are disposed around a periphery of each of the through-holes 11. The first trace 12 is electrically connected to all first contact points 14, and each of the plurality of second traces 13 is electrically connected to the second contact point 15 disposed around the periphery of the through-hole 11 corresponding to the second trace 13.

In some embodiments, the first surface S1 is a front surface of the substrate 10, and the second surface S2 is a back surface of the substrate 10.

In some embodiments, as shown in FIG. 2, the first surface S1 of the substrate 10 is further provided with a signal interface 16. The signal interface 16 includes a first signal interface 161 and a plurality of second signal interfaces 162 arranged in sequence (e.g., along a first direction X). The first signal interface 161 is electrically connected to the first trace 12, and each of the second signal interfaces 162 is electrically connected to one of the second traces 13.

In some embodiments, the biochemical sensing array may further include a plurality of signal access channels. One signal access channel is formed by the first signal interface 161, one of the second signal interfaces 162, and a biochemical sensor connected between the first signal interface 161 and the second signal interface 162.

The biochemical sensor refers to an analytical device that combines a biological recognition element with a physicochemical transducer and is capable of converting a biomolecular interaction into a quantifiable signal (e.g., an electrical signal, an optical signal, etc.). In some embodiments, the biochemical sensor may include an enzyme sensor, an immunosensor, a DNA/nucleic acid sensor, a cell/microbial sensor, a biomimetic sensor, or the like.

In some embodiments, when measuring the biochemical sensing array using a biological detector, the signal interface 16 of the biochemical sensing array is inserted into a card slot of the biological detector, such that the first signal interface 161 and the second signal interfaces 162 are electrically connected to a detection circuit within the biological detector. The first signal interface 161 serves as a common signal interface, and the second signal interfaces 162 serve as detection signal interfaces for detecting the signal of each signal access channel. The signals in the signal access channels may include resistance variation values.

In some embodiments, as shown in FIG. 2, the first surface S1 of the substrate 10 is provided with twenty-five through-holes 11, one first trace 12, and twenty-five second traces 13. The through-holes 11 are arranged in a 5×5 array. Each through-hole 11 is surrounded by one first contact point and one second contact point, thus corresponding to twenty-five groups of first and second contact points, one first trace 12, one first signal interface 161, twenty-five second traces 13, and twenty-five second signal interfaces 162. An end of the first trace 12 is electrically connected to the first signal interface 161, and another end of the first trace 12 is electrically connected to all the first contact points 14. An end of each of the second traces 13 is electrically connected to one of the second signal interfaces 162, and another end of each of the second traces 13 is electrically connected to a corresponding second contact point 15.

In some embodiments, a diameter of each of the through-holes ranges from 2.0 mm to 5.0 mm.

In some embodiments, the diameter of each of the through-holes may be in one of the following ranges: 2.0 mm to 4.0 mm, 2.0 mm to 3.0 mm, 3.0 mm to 5.0 mm, 3.0 mm to 4.0 mm, or the like.

In some embodiments, a distance between centers of adjacent through-holes ranges from 3.0 mm to 15 mm.

In some embodiments, the distance between the centers of adjacent through-holes may be in one of the following ranges: 3.0 mm to 12 mm, 3.0 mm to 10 mm, 3.0 mm to 8 mm, 3.0 mm to 6 mm, 6.0 mm to 15 mm, 6.0 mm to 12 mm, 8.0 mm to 15 mm, 10.0 mm to 15 mm, or the like.

In some embodiments, a distance between an edge of the first contact point and an edge of a corresponding through-hole ranges from 0.3 mm to 1.0 mm.

In some embodiments, the distance between the edge of the first contact point and the edge of the corresponding through-hole may be in one of the following ranges: 0.3 mm to 0.9 mm, 0.3 mm to 0.8 mm, 0.3 mm to 0.7 mm, 0.4 mm to 0.9 mm, 0.5 mm to 0.9 mm, 0.6 mm to 0.9 mm, 0.4 mm to 0.8 mm, 0.3 mm to 0.9 mm, or the like.

In some embodiments, a distance between an edge of each of the second contact points and an edge of a corresponding through-hole ranges from 0.3 mm to 1.0 mm.

In some embodiments, the distance between the edge of each of the second contact points and the edge of the corresponding through-hole may be in one of the following ranges: 0.3 mm to 0.9 mm, 0.3 mm to 0.8 mm, 0.3 mm to 0.7 mm, 0.4 mm to 0.9 mm, 0.5 mm to 0.9 mm, 0.6 mm to 0.9 mm, 0.4 mm to 0.8 mm, 0.3 mm to 0.9 mm, or the like.

In some embodiments, a line width of the first trace ranges from 0.2 mm to 0.5 mm.

In some embodiments, the line width of the first trace may be in one of the following ranges: 0.2 mm to 0.4 mm, 0.2 mm to 0.3 mm, 0.3 mm to 0.5 mm, 0.3 mm to 0.4 mm, or the like.

In some embodiments, a line width of each of the second traces ranges from 0.2 mm to 0.5 mm.

In some embodiments, the line width of each of the second traces may be in one of the following ranges: 0.2 mm to 0.4 mm, 0.2 mm to 0.3 mm, 0.3 mm to 0.5 mm, and 0.3 mm to 0.4 mm.

In some embodiments, a line spacing between the first trace and a second trace adjacent to the first trace ranges from 0.2 mm to 0.5 mm, and a line spacing between adjacent second traces ranges from 0.2 mm to 0.5 mm.

In some embodiments, the line spacing between the first trace and a second trace adjacent to the first trace and the line spacing between adjacent second traces ranges may be in one of the following ranges: 0.2 mm to 0.4 mm, 0.2 mm to 0.3 mm, 0.3 mm to 0.5 mm, 0.3 mm to 0.4 mm, or the like.

In some embodiments, a width of the first signal interface ranges from 0.3 mm to 0.7 mm.

In some embodiments, the width of the first signal interface may be in one of the following ranges: 0.3 mm to 0.6 mm, 0.3 mm to 0.5 mm, 0.4 mm to 0.5 mm, 0.4 mm to 0.6 mm, 0.4 mm to 0.7 mm, or the like.

In some embodiments, a width of the second signal interface ranges from 0.3 mm to 0.7 mm.

In some embodiments, the width of the second signal interface may be in one of the following ranges: 0.3 mm to 0.6 mm, 0.3 mm to 0.5 mm, 0.4 mm to 0.5 mm, 0.4 mm to 0.6 mm, 0.4 mm to 0.7 mm, or the like.

In 120, forming a flexible layer on the second surface by a microelectronic printing technique.

The flexible layer refers to a functional layer made of a deformable material, which may be used to carry a sensing element (e.g., an electrode, a probe, a nanomaterial, etc.) such that sensing element may conform to a non-planar surface and accommodate a dynamic deformation.

The embodiments of the present disclosure do not limit the material of the flexible layer. Any flexible material known to those skilled in the art may be used, such as polydimethylsiloxane (PDMS), polyimide, polyethylene, ethylene-vinyl acetate copolymer, polyvinyl alcohol, polyethylene terephthalate, and polymethyl methacrylate.

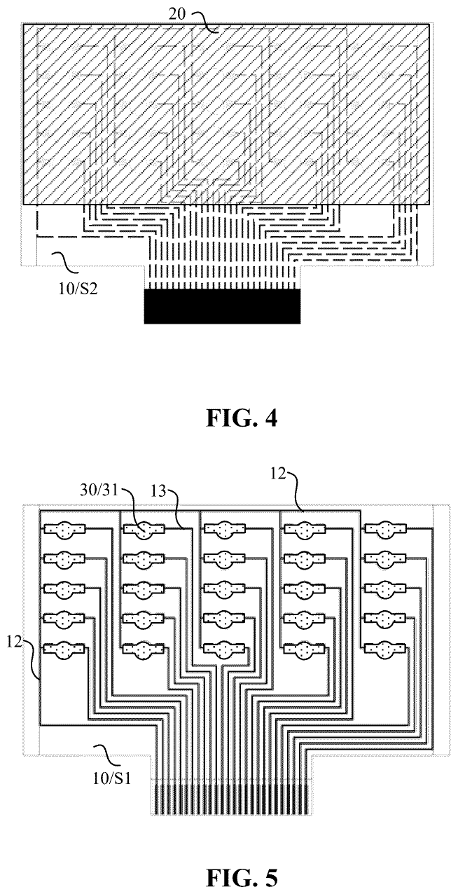

FIG. 4 is a schematic diagram showing an exemplary structure of a flexible layer according to some embodiments of the present disclosure.

In some embodiments, as shown in FIG. 4, the flexible layer (e.g., a flexible layer 20) at least covers the through-holes 11.

In some embodiments, the flexible layer 20 may cover only the through-holes 11, i.e., the flexible layer 20 may not cover a portion of a surface of the substrate 10 surrounding the peripheries of the through-holes 11. In some embodiments, as shown in FIG. 4, the flexible layer 20 covers the through-holes 11 and the portion of the surface (i.e., a portion of the second surface S2) of the substrate 10 surrounding the peripheries of the through-holes 11.

In some embodiments, the flexible layer 20 covers the through-holes 11 and the entire second surface S2 of the substrate 10.

The microelectronic printing method refers to a technique for constructing functional layers (e.g., the flexible layer and the conductive layer) of the sensing array directly on the substrate by layer-by-layer deposition of a functional material (e.g., a polymer, a nano-ink, etc.).

In some embodiments, the microelectronic printing technique for forming the flexible layer on the second surface may include inkjet printing, aerosol jet printing, three-dimensional (3D) printing, dispensing printing, or the like.

In some embodiments, the second surface S2 of the substrate 10 serves as a printing surface. After the substrate 10 is placed in a preset position (e.g., a carrier of a printing device), the flexible layer 20 is formed at a printing location of the flexible layer 20 using the microelectronic printing technique. The printing location of the flexible layer 20 may be set based on a printing coordinate system.

The printing location of the flexible layer 20 includes a printing start position and a printing end position, which respectively refer to positions in the printing coordinate system where the printing starts and ends. The printing location of the flexible layer 20 may be determined based on design requirements.

In some embodiments, the flexible layer includes a polydimethylsiloxane (PDMS) thin-film layer. The PDMS thin-film layer is a flexible and transparent polymer film primarily composed of polydimethylsiloxane.

In some embodiments, forming the flexible layer on the second surface by the microelectronic printing technique includes: obtaining a mixed glue by uniformly mixing a pre-polymer and a curing agent at a mass ratio of M:1, and subjecting the mixed glue to vacuum degassing to remove bubbles, wherein 8≤M≤10; and printing the PDMS thin-film layer through a dispensing technique using the mixed glue as a raw material.

The pre-polymer refers to an oligomer with a certain degree of polymerization that has not yet undergone complete cross-linking. The curing agent refers to a chemical substance capable of initiating or promoting the cross-linking reaction of the pre-polymer. The curing agent may provide a reactive group, a catalyst, or energy (e.g., heat or light) to facilitate the formation of covalent bonds between polymer chains of the pre-polymer, ultimately forming an insoluble and infusible network structure (e.g., the PDMS thin-film layer).

In some embodiments, the pre-polymer may include vinyl-terminated polydimethylsiloxane and a reinforcing filler (e.g., fumed silica), and the curing agent may include a silicon-hydride-containing cross-linking agent and a platinum catalyst.

The dispensing technique refers to a fabrication technique in which a mixture of the pre-polymer and the curing agent is deposited onto the substrate in the form of droplets, lines, or films through precise control of fluid distribution.

In some embodiments, before printing the PDMS thin-film layer, a mixed gel needs to be prepared. The prepared mixed gel is poured into a material cartridge of the printing device, and the second surface S2 of the substrate 10 is placed at the preset position (i.e., the carrier of the printing device). A first printing parameter is configured, and the printing is performed. After printing is completed and the PDMS thin-film layer solidifies, the substrate 10 covered by the PDMS thin-film layer is obtained. The first printing parameter includes the printing location of the flexible layer.

In the embodiments of the present disclosure, by preparing the mixed gel and printing the PDMS thin-film layer using the dispensing technique, the advantages of digital control and flexible processing are combined, enabling high-precision fabrication of PDMS structures to meet manufacturing requirements of the PDMS thin-film layer.

In some embodiments, the first printing parameter further includes a first printing speed and a first printing pressure. The first printing parameter may be determined based on design requirements.

In some embodiments, printing the PDMS thin-film layer using the dispensing technique includes: printing the PDMS thin-film layer based on the first printing speed and the first printing pressure. The first printing speed ranges from 5 mm/s to 15 mm/s, and the first printing pressure ranges from 30 KPa to 90 KPa.

In some embodiments, the first printing speed may be in one of the following ranges: 5 mm/s to 12 mm/s, 5 mm/s to 10 mm/s, 7 mm/s to 15 mm/s, 9 mm/s to 15 mm/s, 6 mm/s to 12 mm/s, or the like.

In some embodiments, the first printing pressure may be in one of the following ranges: 30 KPa to 80 KPa, 30 KPa to 70 KPa, 30 KPa to 60 KPa, 30 KPa to 50 KPa, 40 KPa to 80 KPa, 50 KPa to 80 KPa, 40 KPa to 70 KPa, or the like.

In some embodiments, a thickness of the printed PDMS thin-film layer is negatively related to the first printing speed: the faster the printing speed is, the thinner the PDMS thin-film layer is; the slower the printing speed is, the thicker the layer is. The thickness of the PDMS thin-film layer is positively related to the first printing pressure: the greater the printing pressure is, the thicker the PDMS thin-film layer is; the smaller the printing pressure is, the thinner the layer is.

In some embodiments of the present disclosure, by setting the first printing speed in the range from 5 mm/s to 15 mm/s and the first printing pressure in the range from 30 KPa to 90 KPa, the thickness of the PDMS thin-film layer can be controlled within an appropriate range, balancing sensitivity and impact resistance. If the first printing speed is set above 15 mm/s and/or the first printing pressure is set below 30 KPa, the obtained PDMS thin-film layer may be too thin and prone to cracking or damage. If the first printing speed is set below 5 mm/s and/or the first printing pressure exceeds 90 KPa, the obtained PDMS thin-film layer may be too thick, reducing the deformation of the sensor during the detection of a to-be-tested biomolecule and thereby lowering the sensor's sensitivity.

In some embodiments, after printing the PDMS thin-film layer using the dispensing technique, the method further comprises heating the substrate at a first heating temperature until the PDMS thin film layer solidifies, wherein the first heating temperature ranges from 60° C. to 80° C.

In some embodiments, a heating device (e.g., a hot plate or a drying oven) may be used to heat the substrate at the first heating temperature.

In some embodiments, the first heating temperature may be in one of the following ranges: 60° C. to 70° C., 70° C. to 80° C., 60° C. to 75° C., 65° C. to 80° C., 65° C. to 75° C., or the like.

In some embodiments, the first heating temperature may be one of 65° C., 70° C., 75° C., or the like.

In some embodiments of the present disclosure, the freshly printed PDMS thin-film layer has a degree of adhesiveness. Heating facilitates the rapid solidification of the PDMS thin-film layer, thereby shortening a preparation time and preventing damage to the film layer in subsequent operations.

In some embodiments of the present disclosure, by setting the first heating temperature to 60° C. to 80° C., the solidification time of the PDMS thin-film layer can be shortened as much as possible without compromising the structural integrity of the PDMS thin-film layer. If the first heating temperature is too low (e.g., below 60° C.), the solidification process is slower, resulting in a longer waiting time. Conversely, if the first heating temperature is too high (e.g., above 80° C.), exceeding the melting point of PDMS, bubbles may form in the PDMS thin-film layer, damaging its structure.

It should be noted that any heating device or apparatus known to those skilled in the art may be used to heat the substrate, such as a hot plate or a drying oven, without limitation. A deviation typically exists between a set temperature of the heating device and an actual temperature. While the set temperature is a fixed value, the actual temperature may fluctuate within a predetermined range around the fixed value. For example, if the set temperature is 80° C., the actual temperature may be 80±5° C. As another example, if the set temperature is 60° C., the actual temperature may be 60±3° C.

In 130, forming a conductive layer on the first surface by the microelectronic printing technique.

The conductive layer refers to a functional layer used for transmitting electrical signals.

The embodiments of the present disclosure do not limit the materials used for forming the conductive layer, which may include, but are not limited to, a silver nanowire, a carbon nanotube, conductive silver paste, graphene, a two-dimensional inorganic compound (e.g., MXene), or he like.

FIG. 5 is a schematic diagram illustrating an exemplary structure of a conductive layer according to some embodiments of the present disclosure.

In some embodiments, as shown in FIG. 5, the conductive layer (a conductive layer 30) includes a plurality of separated conductive blocks 31. Each conductive block 31 covers a group (also referred to as a through-hole group) of a through-hole 11 and the first contact point 14 and the second contact point 15 disposed around the periphery of the through-hole.

In some embodiments, as shown in FIG. 5, the substrate 10 is provided with twenty-five through-holes 11. Each of the twenty-five through-holes 11 and the first contact point 14 and the second contact point 15 disposed around the periphery of the through-hole 11 form a group. Accordingly, the conductive layer 30 includes twenty-five separated conductive blocks 31, each covering a group of a through-hole 11 and the first contact point 14 and the second contact point 15 disposed around the periphery of the through-hole 11.

In some embodiments, the first surface S1 of the substrate 10 is the printing surface. The substrate 10 is placed at a preset position (e.g., the carrier of the printing device), and printing locations of the conductive blocks 31 are set based on the printing coordinate system, so that each through-hole group is covered by a conductive block 31. Each of the conductive blocks 31 forms an electrical connection by contacting the first contact point 14 and the second contact point 15, thereby connecting the first trace 11 to the second trace 13 of the sensor.

In some embodiments, the microelectronic printing technique for forming the conductive layer on the first surface may include inkjet printing, nanoimprint printing, laser-induced graphene, dispensing printing, or the like.

In some embodiments, the printing location of each of the conductive blocks 31 includes a printing start position and a printing end position. The printing locations of the conductive blocks 31 may be determined based on design requirements. The processor or the printing device may plan a printing path according to the printing positions and form the conductive layer based on the planned printing path. In some embodiments, the printing positions may further include positions to be covered by the conductive blocks 31, i.e., positions of the through-holes 11, the first contact point 14, and the second contact point 15 in each through-hole group.

In some embodiments, when printing the flexible layer 20 and the conductive layer 30 (or the conductive blocks 31) using a microelectronic printer, the thickness of the printed film may be controlled with nanometer-level precision.

In some embodiments, the conductive layer includes a silver paste layer. Forming the conductive layer on the first surface by the microelectronic printing technique includes: printing the conductive blocks through the dispensing technique using the conductive silver paste as a raw material.

The silver paste layer refers to a conductive layer composed of a conductive material such as silver, an organic binder, a solvent, or the like.

In some embodiments, a conductive silver paste may be poured into the material cartridge of the printing device. The first surface S1 of the substrate 10 is placed on the carrier of the printing device, and a second printing parameter is set before printing. After printing is completed and the silver paste layer solidifies, the substrate 10 covered by a plurality of conductive silver paste blocks is obtained. The second printing parameter includes the printing location of each of the conductive blocks.

In some embodiments, the silver paste conductive blocks exhibit relatively good conductivity. Each silver paste conductive block is in contact with and electrically connected to the first position 14 and the second position 15 in a through-hole group, thereby electrically connecting the first trace 12 and the second trace 13 of the sensor in the through-hole group.

In the embodiments of the present disclosure, for the flexible layer (e.g., the PDMS thin-film layer), the silver paste layer provides an electrical pathway while maintaining flexibility and stretchability.

In some embodiments, the second printing parameter may further include a second printing speed and a second printing pressure. The second printing parameter may be determined based on the design requirements.

In some embodiments, printing the conductive blocks through the dispensing technique includes: printing the conductive blocks using the dispensing technique based on the second printing speed and the second printing pressure, wherein the second printing speed ranges from 4 mm/s to 15 mm/s, and the second printing pressure ranges from 70 kPa to 100 kPa.

In some embodiments, the second printing speed may be in one of the following ranges: 4 mm/s to 12 mm/s, 4 mm/s to 10 mm/s, 4 mm/s to 8 mm/s, 6 mm/s to 15 mm/s, 8 mm/s to 15 mm/s, 10 mm/s to 15 mm/s, 6 mm/s to 12 mm/s or the like.

In some embodiments, the second printing speed may be one of 4 mm/s, 5 mm/s, 10 mm/s, or the like.

In some embodiments, the second printing pressure may be in one of the following ranges: 70 kPa to 100 kPa, 70 kPa to 90 kPa, 70 kPa to 80 kPa, 80 kPa to 100 kPa, 90 kPa to 100 kPa, 80 kPa to 90 kPa, or the like.

In some embodiments, the second printing pressure may alternatively be 80 kPa or 100 kPa.

In some embodiments, a thickness of each of the printed conductive blocks 31 is negatively correlated with the second printing speed: the faster the second printing speed is, the thinner the conductive block 31 is; the slower the second printing speed is, the thicker the conductive block is. The thickness of each of the conductive blocks 31 is positively correlated with the second printing pressure: the higher the second printing pressure is, the thicker the conductive block is; the lower the second printing pressure is, the thinner the conductive block is.

In some embodiments of the present disclosure, by setting the second printing speed to 4 mm/s to 15 mm/s and the second printing pressure to 70 kPa to 100 kPa, the thickness of the conductive blocks can be controlled within a suitable range, balancing reliability of the sensor and a fabrication cost. If the second printing speed is set higher than 15 mm/s and/or the second printing pressure is lower than 70 kPa, the obtained conductive blocks 31 may be too thin, causing disconnection between the first trace 12 and the second trace 13, thereby compromising sensor reliability. If the second printing speed is lower than 4 mm/s and/or the second printing pressure is greater than 100 kPa, the obtained conductive blocks 31 may be too thick, which increases the fabrication cost.

In some embodiments, after printing the conductive blocks using the dispensing technique, the method further includes: heating the substrate at a second heating temperature for a heating duration until the conductive blocks solidify, wherein the second heating temperature ranges from 80° C. to 120° C., and the heating duration ranges from 30 minutes to 50 minutes.

In some embodiments, a heating device may be used to heat the substrate at the second heating temperature.

In some embodiments, the second heating temperature may be in one of the following ranges: 80° C. to 110° C., 80° C. to 100° C., 80° C. to 90° C., 90° C. to 120° C., 100° C. to 120° C., 90° C. to 110° C., or the like.

In some embodiments, the second heating temperature may be one of 80° C., 100° C., 120° C., or the like.

In some embodiments, the heating duration may be in one of the following ranges: 30 to 40 minutes, 40 to 50 minutes, 35 to 50 minutes, 35 to 45 minutes, 30 to 45 minutes, or the like.

In some embodiments, the heating duration may be one of 30 minutes, 40 minutes, 50 minutes, or the like.

The second heating duration is related to a thickness of the silver paste layer and the second heating temperature. Under a fixed thickness of the silver paste layer, the higher the second heating temperature, the shorter the required second heating duration for the silver paste layer to solidify; conversely, the lower the second heating temperature, the longer the required second heating duration.

In the embodiments of the present disclosure, the freshly printed silver paste layer still exhibits a degree of fluidity. Heating may accelerate the solidification of the silver paste layer, thereby shortening the preparation time and preventing subsequent operations from affecting the uniformity of the thickness of the layer or damaging a structure of the conductive blocks. By setting the second heating temperature to 80° C. to 120° C., the solidification of the silver paste layer can be accelerated without affecting the solidified PDMS thin-film layer.

It should be noted that any device with a heating function known to those skilled in the art, such as a heating stage or a drying oven, may be used to heat the substrate. The present disclosure does not limit the types of heating devices. A deviation may exist between a set temperature and an actual temperature of the heating device. The set temperature refers to a fixed temperature value, while the actual temperature may fluctuate within a preset range around the fixed temperature value. For example, if the set temperature is 100° C., the actual temperature may be 100±5° C. As another example, if the set temperature is 90° C., the actual temperature may be 90±5° C.

In 140, forming a sensitive layer a side of the flexible layer away from the substrate.

The sensitive layer refers to a core functional layer in the biochemical sensor that is responsible for a specific interaction with a target molecule (e.g., glucose, a protein, a DNA, an ion, a gas, etc.) and converting a biochemical signal into a detectable physical signal (e.g., an electrical signals).

In some embodiments, the sensitive layer may be composed of a biorecognition element and a signal transduction material.

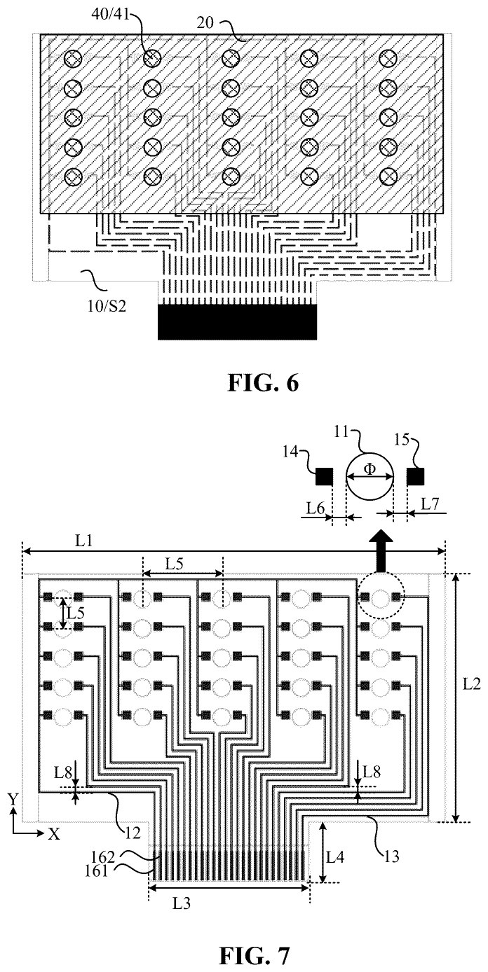

FIG. 6 is a schematic diagram illustrating an exemplary structure of a sensitive layer according to some embodiments of the present specification.

In some embodiments, as shown in FIG. 6, the sensitive layer (e.g., a sensitive layer 40) includes a plurality of separated thin-film blocks 41, and each of the thin-film blocks 41 at least partially overlaps with one of the through-holes 11.

Each of the thin-film blocks 41 at least partially overlaps with one of the through-holes 11 refers to that a projection of each of the thin-film blocks 41 in a direction perpendicular to the substrate partially overlaps with a projection of one of the through-holes 11 in the direction perpendicular to the substrate.

In some embodiments, as shown in FIG. 6, twenty-five through-holes 11 are provided on the substrate 10, and the sensitive layer 40 includes twenty-five separated thin-film blocks 41, each of which at least partially overlap with one of the twenty-five through-holes 11.

In some embodiments, forming the sensitive layer on the side of the flexible layer facing away from the substrate includes: aligning a mask plate with the second surface S2 of the substrate 10, wherein the mask plate is provided with through-holes arranged in an array, each of which at least partially overlaps with one of the through-holes 11 on the substrate 10; then forming the sensitive layer 40 on a side of the mask plate away from the substrate 10; after removing the mask plate, a plurality of separated thin-film blocks 41 are formed on the side of the flexible layer 20 away from the through-holes 11.

In some embodiments, the substrate 10 in operation 110 may be used as the mask plate, and the substrate 10 is placed over another substrate 10 having the flexible layer 20, such that the through-holes 11 of the two substrates 10 are aligned.

In some embodiments, operations 130 and 140 may be performed in reverse order, i.e., the sensitive layer 40 may be formed before the conductive layer 30.

It should be noted that FIGS. 2 to 6 illustratively show that the biochemical sensing array includes twenty-five biochemical sensors arranged in a 5×5 array, and that shapes of the first point 14 and the second point 15 are square. However, this should not be construed as limiting the method for preparing the biochemical sensing array provided in the embodiments of the present disclosure. In other embodiments, the biochemical sensing array may include fewer or more biochemical sensors, such as thirty-two or twenty biochemical sensors. The first point 14 and the second point 15 may also be configured in other shapes, such as rectangular, circular, or triangular, without limitation.

In some embodiments, the sensitive layer includes a nano-gold layer, and forming the sensitive layer on the side of the flexible layer away from the substrate includes forming the thin-film blocks on the side of the flexible layer facing away from the substrate by a magnetron sputtering technique.

The nano-gold layer refers to an ultrathin functional layer composed of gold nanoparticles (AuNPs) or a gold nanostructure, which possesses unique physicochemical properties that enable efficient detection of a target substance (e.g., a biomolecule, a gas, a chemical, etc.).

In some embodiments, a material of the nano-gold layer is high-purity gold, and a particle size of each of the gold nanoparticles may reach a nanometer scale. For example, the size of the gold nanoparticles or the gold nanostructure in the nano-gold layer may range from 1 nm to 100 nm.

In some embodiments, the prepared nano-gold layer includes a plurality of separated thin-film blocks 41, and each of the thin-film blocks 41 at least partially overlaps with one of the through-holes 11.

The magnetron sputtering technique is a type of physical vapor deposition (PVD) technique that uses a magnetic field to confine plasma, whereby high-energy particles bombard a target material to sputter its atoms or molecules, which then deposit onto a substrate surface (e.g., PDMS, silicon wafer, etc.).

In some embodiments, the magnetron sputtering technique may be performed using a magnetron sputtering device (e.g., a magnetron sputtering instrument) with a magnetron sputtering parameter applied to the substrate.

In some embodiments, the magnetron sputtering parameter includes at least a first sputtering duration. The first sputtering duration refers to a duration from a starting time point of sputtering the target material to an ending time point of power input, and may be controlled by the magnetron sputtering device. The sputtering parameter may be determined based on design requirements.

In some embodiments of the present disclosure, by preparing the nano-gold layer using the magnetron sputtering technique, a thickness and uniformity of the nano-gold layer can be precisely controlled. The obtained nano-gold layer is a high-purity, dense, and uniform film with strong adhesion to the flexible layer and exhibits a high mechanical strength. The nano-gold layer also offers advantages such as a high sputtering rate, simple operation, low working temperature, low energy consumption, and environmental friendliness. In addition, the nano-gold layer exhibits excellent biocompatibility and can bind to a variety of biomolecules without affecting their biological activity.

In some embodiments, forming the thin-film blocks on the side of the flexible layer away from the substrate by the magnetron sputtering technique includes: sputtering thin-film blocks on the side of the flexible layer away from the substrate based on a first sputtering duration, wherein the first sputtering duration ranges from 1 minutes to 2 minutes.

In some embodiments, the first sputtering duration may be in one of the following ranges: 1 min to 1.5 min, 1.5 min to 2 min, 1.25 min to 2 min, 1 min to 1.75 min, or the like.

In some embodiments, the first sputtering duration is positively correlated with a thickness of the nano-gold layer. The longer the first sputtering duration is, the thicker the nanogold layer is; the shorter the first sputtering duration is, the thinner the nano-gold layer is.

In some embodiments of the present disclosure, the thickness of the nano-gold layer may be controlled by adjusting the first sputtering duration. By keeping the first sputtering duration within the range of 1 minute to 2 minutes, the binding performance between the nano-gold layer and antibodies can be ensured, while also preventing the nano-gold layer from becoming excessively thick, which may otherwise increase cost.

Merely by way of example, the method for preparing the biochemical sensing array includes the following steps:
(1) Preparing a PDMS mixed glue. A component A (e.g., the pre-polymer) and a component B (e.g., the curing agent) may be mixed at a mass ratio of 10:1 and stirred thoroughly to obtain a mixed glue, which is then subjected to vacuum degassing to remove bubbles.
(2) Printing the PDMS film layer. The prepared PDMS mixture is poured into the material cartridge of a microelectronic printer, a backside of the flexible substrate is placed on the printer stage, the first printing speed is set to 8 mm/s and the first printing pressure is set to 90 kPa. After printing is completed, the flexible substrate is heated at 80° C. until the PDMS thin-film layer solidifies, thereby obtaining a flexible substrate with the PDMS thin-film layer covering its backside.
(3) Printing the silver paste layer. A conductive silver paste is poured into the material cartridge of the microelectronic printer, a front side of the flexible substrate is placed on the printer stage, the second printing speed is set to 5 mm/s and the second printing pressure is set to 80 kPa. After printing is completed, the flexible substrate is heated at 120° C. for 30 minutes to obtain a flexible substrate with the conductive layer covering its front side.
(4) Forming the nano-gold layer. The nano-gold layer is sputtered on a surface of the PDMS thin-film layer using the magnetron sputtering instrument to a gold nanoparticles-polydimethylsiloxane (AuNPs-PDMS) composite film.

In some embodiments, the first printing speed for printing the PDMS thin-film layer may be set to 6 mm/s, and the first printing pressure may be set to 80 kPa.

In some embodiments, the second printing speed for printing the silver paste layer may be set to 4 mm/s, and the second printing pressure may be set to 100 kPa.

In some embodiments, the method for preparing the biochemical sensing array further includes: modifying an antibody on a side of each of the thin-film blocks away from the flexible layer, wherein the antibody is configured to bind specifically with the to-be-tested biomolecule.

In some embodiments of the present disclosure, the modification of the antibody is achieved by dropping an antibody solution onto each of the thin-film blocks 41 of the flexible layer 20 and maintaining the antibody solution for a predetermined duration, thereby immobilizing antibody molecules onto the thin-film blocks 41. Different concentrations of to-be-tested biomolecule solutions are respectively dropped onto different biochemical sensors, allowing the to-be-tested biomolecule to specifically bind with the antibody. The flexible layer 20 and the sensitive layer 40 undergo deformation, which increases a distance between conductive silver paste segments in the conductive blocks, reduces conductive paths, and increases resistance of the sensor. The resistance may be measured using a biosensor detector, thereby enabling detection of the to-be-tested biomolecule.

In some embodiments of the present disclosure, the sensitive layer 40 is the nano-gold layer. The antibody solution is dropped onto each thin-film block of the nano-gold layer and maintained for a preset duration, allowing antibody molecules to be immobilized onto the nano-gold layer. The biochemical sensing array is then rinsed with a buffer solution to suppress nonspecific adsorption of the antibody molecules.

In some embodiments of the present disclosure, the microelectronic printing technique offers the advantage of an adjustable printing parameter, enabling precise control over film thickness and standardized film fabrication, thereby facilitating the standardized preparation of biochemical sensors. The obtained biochemical sensing array is a surface stress-based biochemical sensor comprising a plurality of biochemical sensors arranged in an array, with each through-hole corresponding to one biochemical sensor. The biochemical sensors exhibit good uniformity and reproducibility, making them suitable for high-throughput detection. Compared with individual biochemical sensors, this configuration significantly improves detection accuracy and efficiency.

In some embodiments of the present disclosure, the method for preparing the biochemical sensing array uses environmentally friendly raw materials, avoiding the use of toxic substances such as fluorescent reagents and thus preventing environmental pollution, aligning with the green development philosophy. Furthermore, quantitative analysis and scientific evaluation of potential ecological benefits indicate that the method significantly contributes to sustainable economic development and efficient resource utilization.

In some embodiments, the biochemical sensing array includes a plurality of biochemical sensors corresponding to the through-holes, and at least two of the biochemical sensors are different. The sensitive layer further includes a microfluidic channel and a target sample region, and the microfluidic channel is in communication with the target sample region and the plurality of biochemical sensors.

In some embodiments, the method further includes: determining a microfluidic channel feature based on antibody information of the plurality of biochemical sensors and dimensional information of the through-holes; and fabricating the microfluidic channel using the microelectronic printing technique based on the microfluidic channel feature.

The biochemical sensors are disposed within the through-holes, with each biochemical sensor corresponding to one of the through-holes.

The microfluidic channel refers to a printed fluid channel, such as a groove formed by a printed pattern. The microfluidic channel is disposed between the sensitive layer and the flexible layer, and a sample (e.g., the to-be-tested biomolecule) may flow from the target sample region into the biochemical sensors through the microfluidic channel. If the sensitive layer only covers the through-holes, the microfluidic channel may be fabricated simultaneously with the sensitive layer. In other cases, the microfluidic channel may be fabricated after the sensitive layer has been formed.

The target sample region refers to a region where the to-be-tested biomolecule is dropped. The target sample region may be located in regions of the sensitive layer that do not contain through-holes.

The antibody information of a biochemical sensor refers to information about the antibody molecules immobilized on the film blocks. The antibody information includes a type and a concentration of the antibody. Different sensors may have different antibody information (e.g., different antibody concentrations).

The dimensional information of the through-holes refers to information such as a size, shape, and an arrangement of the through-holes. The arrangement may be stored in a vector database in the form of X×Y, representing X rows and Y columns.

The microfluidic channel feature refers to a physical feature of the microfluidic channel. In some embodiments, the microfluidic channel feature may include a size (e.g., a width and a depth), a shape, and a flow direction of the microfluidic channel. The shape may include a cross-sectional shape (e.g., semicircular, arc-shaped, etc.) and a top view shape (e.g., spiral, linear, etc.). The flow direction refers to a direction in which the sample (e.g., the to-be-tested biomolecule) flows between biochemical sensors from the target sample region.

In some embodiments, the processor may determine the microfluidic channel feature based on the antibody information of the biochemical sensors and the dimensional information of the through-holes, using a vector database.

In some embodiments, the processor may construct a feature vector based on the antibody information and the dimensional information of the through-holes. The processor determines the microfluidic channel feature based on a retrieval result for the feature vector from the vector database. The vector database includes a plurality of reference vectors and a microfluidic channel feature corresponding to each of the reference vectors. The reference vectors are constructed based on experimental data. The processor may determine a vector distance between the feature vector and each of the reference vectors and select the microfluidic channel feature corresponding to the reference vector with a minimum distance to the feature vector.

In some embodiments, the processor may construct the reference vectors based on the antibody information and the dimensional information of the through-holes. Then, for each of the reference vectors, under the conditions of the antibody information and the dimensional information of the through-holes for determining the reference vector, a technician may use computational fluid dynamics (CFD) software to simulate multiple sets of randomly generated initial microfluidic channel features. Microfluidic channel features that enable the sample to demonstrate relatively good flow performance (e.g., the sample exhibits a flowability above a threshold) are selected from a simulation result for sensor testing, and a microfluidic channel feature that meets a sensor requirement (e.g., sensitivity of the sensor is above a threshold) are recorded as the microfluidic channel feature corresponding to the reference vector.

In some embodiments, the vector database further includes fabrication parameters corresponding to the microfluidic channel features, which may be used to fabricate the microfluidic channel via the microelectronic printing technique.

In some embodiments of the present disclosure, by determining the microfluidic channel feature, fluid dynamics can be utilized to guide directional flow of the sample, which allows the biochemical sensing array to be more versatile, enabling the simultaneous detection of multiple biochemical signals.

In some embodiments, the microfluidic channel includes a plurality of sub-channels that are in communication with a plurality of biochemical sensors. A size-selective micropillar array is provided upstream of the sub-channels, and a dimension of the size-selective micropillar array is related to the antibody information of the biochemical sensors connected to the microfluidic channel. A spiral microchannel is provided downstream of the sub-channels.

The size-selective micropillar array refers to an array designed for filtering molecules of different sizes. The size-selective micropillar array may be integrated upstream of the sub-channels. A spacing between micropillars in the size-selective micropillar array is larger than a diameter of a target molecule (e.g., the to-be-tested biomolecule) and smaller than diameters of non-target molecules, thus enabling physical interception of large particles and allowing only the to-be-tested biomolecule to pass through.

The spiral microchannel refers to a channel shaped in a spiral configuration. By configuring a width of the spiral microchannel to decrease gradually, a Dean vortex effect and an inertial lifting force can be leveraged to focus large particles (e.g., cells, debris) toward an outer edge of the spiral microchannel, while the to-be-tested biomolecule or proteins with a relatively small diameter remain near a central streamline of the spiral microchannel and flow directly toward the through-holes or the biochemical sensors.

In some embodiments, a relationship between the dimension of the size-selective micropillar array and the antibody information may be stored in a predefined mapping table.

In some embodiments, the microfluidic channel feature further includes characteristics of the size-selective micropillar array and the spiral microchannel.

In some embodiments of the present disclosure, by providing the size-selective micropillar array and the spiral microchannel, particle separation and inertial focusing effects in the spiral microchannel can reduce interference with the biochemical sensors by other molecules.

In some embodiments, the microfluidic channel includes one or more microvalves configured to adjust the flow direction of the microfluidic channel.

In some embodiments, the one or more microvalves may be deformable membrane(s). The deformable membranes may undergo deformation in response to an externally applied force such as an electrostatic force, an electromagnetic force, thermal expansion, or the like, thereby blocking or opening a region of the microfluidic channel to achieve a valve function.

In some embodiments, the one or more microvalves may be electrowetting valve(s). By applying a voltage to change a contact angle of a liquid on a hydrophobic surface, the electrowetting valve(s) may control a movement of the liquid or form a droplet barrier.

In some embodiments, the microfluidic channel may have a plurality of different flow directions.

For example, when microvalve 1 is open and microvalve 2 is closed, the flow direction is opposite to the scenario where microvalve 1 is closed and microvalve 2 is open. The plurality of subchannels have different flow directions and/or flow paths when different microvalves are opened or closed.

In some embodiments of the present disclosure, by configuring the one or more microvalves, the flow direction of the microfluidic channel can be adjusted to guide the directional flow of the sample.

In some embodiments, during a trial manufacturing stage of the biochemical sensing array, a target opening/closing state of each of the one or more microvalves is determined based on an operating mode and a real-time resistance value of the biochemical sensing array.

The operating mode of the biochemical sensing array may include a cleaning mode and a detection mode. The cleaning mode is configured to flush out impurities from the biochemical sensing array, and the detection mode is configured to detect biomolecules. In the cleaning mode, all microvalves in the microfluidic channel are open; in the detection mode, a portion of the microvalves in the microfluidic channel are closed. In some embodiments, the operating mode may be preset.

The real-time resistance value refers to the resistance of the biochemical sensing array detected by a bio-detector in real time. Under the corresponding operating mode (the cleaning mode or the detection mode), if the real-time resistance value does not meet expectations, the target opening/closing state of each of the one or more microvalves may be adjusted.

During the fabrication of the plurality of biochemical sensors, the thickness of the nano-gold layer may vary, leading to a difference in the binding performance of the biochemical sensors with the antibody. Therefore, different biochemical sensors may have different accuracies. If the real-time resistance value does not match an actual sample concentration, the target opening/closing state of each of the one or more microvalves may be adjusted accordingly. For example, if the sample concentration is high but the real-time resistance value is relatively low, the microvalve corresponding to the biochemical sensor with a relatively thick nano-gold layer may be opened.

After determining the target opening/closing state of each of the one or more microvalves, the flow direction of the microfluidic channel may be determined based on the target opening/closing state and a corresponding thickness of the nano-gold layer for fabricating the microfluidic channel. In a final usage phase of the biochemical sensing array, the target opening/closing state of each of the one or more microvalves is no longer adjusted. Merely by way of example, during the trial manufacturing stage, it may be experimentally determined that biochemical sensor A exhibits the highest sensitivity when the thickness of the nano-gold layer is H1. Therefore, for a biochemical sensing array composed entirely of biochemical sensors A, the microfluidic channel may be prepared based on the thickness H1 of the nano-gold layer and a configuration of the microfluidic channel that corresponds to the highest accuracy of the biochemical sensor A, so as to improve the accuracy of the biochemical sensing array and reduce the thickness of the nano-gold layer, thereby reducing the manufacturing cost.

In some embodiments of the present disclosure, the target opening/closing state of each of the one or more microvalves is determined by the operation mode and the real-time resistance value, allowing for the use of the biochemical sensors with more appropriate accuracies. In a test phase of the biochemical sensing array, by measuring the thickness of the nano-gold layer, the biochemical sensors with the highest sensitivity and the thinnest nano-gold layer can be obtained, thereby reducing the overall manufacturing cost of the biochemical sensing array.

In some embodiments, the method further includes: determining a target magnetron sputtering parameter based on the microfluidic channel feature and a preset magnetron sputtering parameter, and preparing the nano-gold layer using the magnetron sputtering instrument based on the target magnetron sputtering parameter. The preset magnetron sputtering parameter corresponds to the antibody information of the biochemical sensors and the dimensional information of the through-holes.

In some embodiments, if the microfluidic channel feature is determined after the magnetron sputtering technique is performed, there may be an effect on the nano-gold layer that has been subjected to the magnetron sputtering technique. Thus, the effect may be taken into account in advance during magnetron sputtering to prepare the nano-gold layer.

The preset magnetron sputtering parameter refers to a preset parameter for performing the magnetron sputtering technique without considering the microfluidic channel. In some embodiments, the preset magnetron sputtering parameter includes a first sputtering duration, a sputtering power, a substrate temperature, or the like. The higher the sputtering power, the faster a deposition rate is, which may result in increased stress of the thin-film blocks or changes in grain size. The substrate temperature refers to a temperature of a substrate holder of the magnetron sputtering instrument. The substrate holder is capable of performing heating, cooling, rotating, biasing, or the like.

In some embodiments, the preset magnetron sputtering parameter may be preset based on experience or experimentation. The preset magnetron sputtering parameters of different biochemical sensors may vary due to different antibody information of the different biochemical sensors and different dimensional information of the through-holes.

The target magnetron sputtering parameter refers to a parameter with which the magnetron sputtering device (e.g., the magnetron sputtering instrument) performs magnetron sputtering.

In some embodiments, the processor may determine the target magnetron sputtering parameter based on the microfluidic channel feature and the preset magnetron sputtering parameter through a parameter determination model.

In some embodiments, the parameter determination model may be a machine learning model. For example, the parameter determination model may be a neural network (NN) model, a recurrent neural network (RNN) model, etc.

In some embodiments, an input of the parameter determination model may include the microfluidic channel feature and the preset magnetron sputtering parameter, and an output of the parameter determination model may include the target magnetron sputtering parameter.

In some embodiments, the parameter determination model may be obtained by training based on preferred historical data.

The preferred historical data may be obtained by evaluating historical data with a uniformity score and a thickness score. Uniformity refers to a standard deviation of resistance values of identical biochemical sensors. The smaller the standard deviation is, the higher the uniformity score is. The thickness score refers to a score of the thickness of the nano-gold layer. The smaller the thickness of the nano-gold layer is, the higher the thickness score is. Historical data with the highest weighted sum of the uniformity score and the thickness score may be selected as the preferred historical data. Alternatively, historical data with a uniformity score exceeding a uniformity threshold and a thickness score exceeding a thickness threshold may be selected as the preferred historical data.

In some embodiments, the processor may obtain a plurality of training samples with labels to form a training sample set. The training samples may include sample microfluidic channel features and sample preset magnetron sputtering parameters. The labels may be actual magnetron sputtering parameters corresponding to the training samples.

In some embodiments, the processor may input the training sample set into an initial parameter determination model to perform a plurality of rounds of iterations. Each round of iterations includes selecting one or more training samples from the training sample set, inputting the one or more training samples into the initial parameter determination model, obtaining one or more model prediction outputs corresponding to the one or more training samples; substituting one or more model prediction outputs and the labels of the one or more training samples into a predefined loss function to determine a value of the loss function; iteratively updating model parameters of the initial parameter determination model based on the value of the loss function. When an iteration end condition is satisfied, the iteration is ended to obtain the trained prediction model. The iteration end condition may include convergence of the loss function, a count of the iteration reaching a threshold, etc.

In some embodiments of the present disclosure, determining the target magnetron sputtering parameter by the parameter determination model can utilize a self-learning capability of the machine learning model to identify patterns from a large amount of historical data, which improves the accuracy and efficiency of determining the target magnetron sputtering parameter.

It should be noted that the foregoing descriptions of process 100 are intended to be exemplary and illustrative only and do not limit the scope of application of the present disclosure. For those skilled in the art, various corrections and changes may be made to process 100 under the guidance of the present disclosure. However, these corrections and changes remain within the scope of the present disclosure.

On the basis of the above embodiments, one or more embodiments of the present disclosure further provide a biochemical sensing array, which is prepared through the method for preparing a biochemical sensing array as described in any one of the embodiments of the present disclosure, and has corresponding beneficial effects. The common aspects may be understood with reference to the above descriptions.

In some embodiments, as shown in FIGS. 2 to 6, the biochemical sensing array includes a substrate 10, a flexible layer 20, a conductive layer 30, and a sensitive layer 40.

In some embodiments, the substrate 10 includes a first surface S1 and a second surface S2 disposed opposite to each other, the first surface S1 is provided with a plurality of through-holes 11 arranged in an array, a first trace 12, and a plurality of second traces 13, each of the plurality of second traces 13 corresponding to one of the through-holes 11, a first contact point 14 and a second contact point 15 disposed around the periphery of each of the through-holes 11, the first trace 12 is electrically connected to all first contact points 14, and each of the second traces 13 is electrically connected to the second contact point 15 disposed around the periphery of the through-hole 11 corresponding to the second trace 13.

In some embodiments, the flexible layer 20 is disposed on the second surface S2 of the substrate 10, covering at least the through-holes 11.

In some embodiments, the sensitive layer 40 is disposed on a side of the flexible layer 20 away from the substrate 10, and the sensitive layer 40 includes a plurality of separated thin-film blocks 41, each of the thin-film blocks 41 at least partially overlapping with one of the through-holes 11.

In some embodiments, the conductive layer 30 is disposed on the first surface S1 of the substrate 10. The conductive layer 30 includes a plurality of separated conductive blocks 31, each conductive block 31 covering a group of a through-hole 11 and a first contact point 14 and a second contact point 15 disposed around the periphery of the through-hole 11. Each of the conductive blocks 31 contacts the first contact point 14 and the second contact point 15 covered by the conductive block 31 to form an electrical connection, thereby conducting the first trace 11 and the second trace 13 of the biochemical sensor (i.e., the group of the through-hole 11 and the first contact point 14 and the second contact point 15 disposed around the periphery of the through-hole 11).

In some embodiments, a count of the biochemical sensors of the biochemical sensing array is equal to a count of the through-holes 11. In some embodiments, as shown in any one of FIGS. 2 to 6, the biochemical sensing array includes twenty-five biochemical sensors, and the count of the through-holes 11 is twenty-five.

It should be noted that FIGS. 2-6 exemplarily illustrate that the biochemical sensing array includes twenty-five biochemical sensors arranged in a 5×5 array, and each of the first contact point 14 and the second contact point 15 is shaped as a square. However, this configuration does not constitute a limitation of the biochemical sensing array provided by the embodiments of the present disclosure. In other embodiments, the biochemical sensing array may be provided with a smaller or a greater count of biochemical sensors. For example, the count of the biochemical sensors may be thirty-two or twenty, and the first contact point 14 and the second contact point 15 may be configured in other shapes, for example, a rectangular, a circular, or a triangular, which is not limited herein.

FIG. 7 is a schematic diagram illustrating an exemplary structure of a substrate according to some embodiments of the present disclosure.

In some embodiments, as shown in FIG. 7, along a first direction X, a length L1 of the substrate 10 is 70 mm, the length L3 of a signal interface of the first surface S1 is 28.5 mm, a distance L5 between centers of adjacent through-holes 11 is 13.4 mm, a distance L6 between an edge of the first contact point 14 and an edge of a corresponding through-hole 11 is 0.5 mm, and a distance L7 between an edge of each of the second contact points 14 and an edge of a corresponding through-hole 11 is 0.5 mm. Along a second direction Y, a width L2 of an array distribution region (i.e., the region where the biochemical sensors are distributed) of the substrate 10 is 42 mm, and a width L4 of a trace tail region (i.e., the region where the tails of the traces are located) is 10 mm; a distance L5 between the centers of adjacent through-holes 11 is 5 mm; a line spacing L8 between the first trace 12 and a second trace 13 adjacent to the first trace 12 is 0.5 mm, a line spacing L8 between adjacent second traces is 0.5 mm; a diameter Φ of each of the through-holes 11 is 3 mm; a line width of the first trace and a line width of each of the second traces are 0.3 mm. Along the first direction X, a width of the first signal interface 161 is 0.5 mm, and a width of each of the second signal interfaces 162 is 0.5 mm.

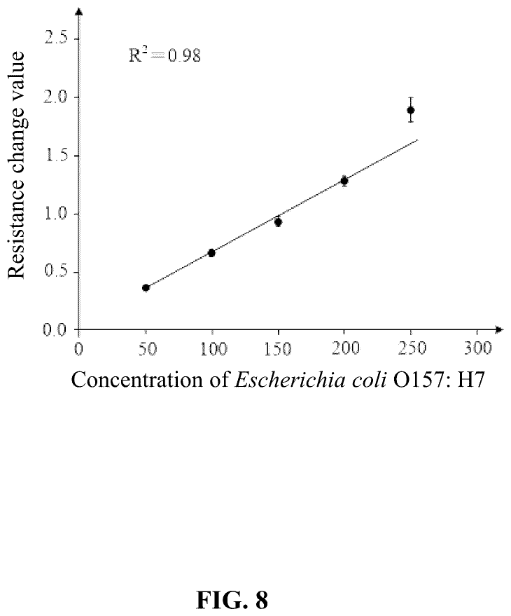

FIG. 8 is an exemplary schematic diagram illustrating a detection result of a biochemical sensing array according to some embodiments of the present disclosure.

In some embodiments, an exemplary detection process for Escherichia coli (E. coli) O157:H7 using the biochemical sensing array provided in the embodiments of the present disclosure includes:
(1) Diluting the E. coli O157:H7 to concentrations of 50 Colony-Forming Units per Milliliter (CFU/mL), 100 CFU/mL, 150 CFU/mL, 200 CFU/mL, and 250 CFU/mL, respectively.
(2) Dropwise adding the above E. coli O157:H7 solutions at different concentrations to the biochemical sensors and reacting for 30 minutes to allow specific binding of the E. coli O157:H7 to the antibodies.
(3) Measuring resistance values using a portable in vitro bio-detector. As shown in FIG. 5, the biochemical sensing array has twenty-six signal interfaces: the leftmost signal interface is the first signal interface (i.e., a common signal interface), and the remaining 25 are the second signal interfaces (i.e., measurement signal interfaces). An impedance testing zone exists between each second signal interface and the first signal interface. If an impedance of the solution in the testing zone changes, the measured resistance between the two signal interfaces also changes.
(4) The detection result is shown in FIG. 8, a resistance change value is linearly related to the concentration of the E. coli O157:H7 solution, and the resistance change value increases with the concentration of the E. coli O157:H7 solution. A variance R² of a fitted formula is 0.98, which indicates that the biochemical sensing array has good uniformity and repeatability.

Based on the above embodiments, one or more embodiments of the present disclosure further provide a test strip including the biochemical sensing array as described above. The biochemical sensing array has the corresponding beneficial effects, which are not repeated herein.

Based on the above embodiments, one or more embodiments of the present disclosure further provide a test kit including the test strip as described above. The test strip has the corresponding beneficial effects, which are not repeated herein.

In other embodiments, the test kit may further include all of the constituent structures known to those skilled in the art, such as a housing, a detection reagent, a dropper, a ziplock bag, or the like, which are not limited herein.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment,” “one embodiment,” or “an alternative embodiment” in various portions of the present disclosure are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations, therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software-only solution, e.g., an installation on an existing server or mobile device.

Similarly, it should be appreciated that in the foregoing description of embodiments of the present disclosure, various features are sometimes grouped together in a single embodiment, figure, or description thereof to streamline the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities, properties, and so forth, used to describe and claim certain embodiments of the application are to be understood as being modified in some instances by the term “about,” “approximate,” or “substantially.” For example, “about,” “approximate” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A method for preparing a biochemical sensing array, comprising:
providing a substrate, wherein the substrate includes a first surface and a second surface disposed opposite to each other, the first surface is provided with through-holes arranged in an array, a first trace, and a plurality of second traces; each of the plurality of second traces corresponds to one of the through-holes, a first contact point and a second contact point are disposed around a periphery of each of the through-holes, the first trace is electrically connected to all first contact points, and each of the plurality of second traces is electrically connected to the second contact point disposed around the periphery of the through-hole corresponding to the second trace;
forming a flexible layer on the second surface by a microelectronic printing technique, wherein the flexible layer at least covers the through-holes;
forming a conductive layer on the first surface by the microelectronic printing technique, wherein the conductive layer includes a plurality of separated conductive blocks, each conductive block covering a group of a through-hole and the first contact point and the second contact point disposed around the periphery of the through-hole; and
forming a sensitive layer on a side of the flexible layer away from the substrate, wherein the sensitive layer includes a plurality of separated thin-film blocks, each of the thin-film blocks at least partially overlapping with one of the through-holes.

2. The method of claim 1, wherein the flexible layer includes a polydimethylsiloxane (PDMS) thin-film layer; and the forming a flexible layer on the second surface by a microelectronic printing technique includes:
obtaining a mixed glue by uniformly mixing a pre-polymer and a curing agent at a mass ratio of M:1, and subjecting the mixed glue to vacuum degassing to remove bubbles, wherein 8≤M≤10; and
printing the PDMS thin-film layer through a dispensing technique using the mixed glue as a raw material.

3. The method of claim 2, wherein the printing the PDMS thin-film layer through a dispensing technique includes:
printing the PDMS thin-film layer through the dispensing technique based on a first printing speed and a first printing pressure, wherein the first printing speed ranges from 5 mm/s to 15 mm/s, and the first printing pressure range from 30 kPa to 90 kPa.

4. The method of claim 3, wherein after printing the PDMS thin-film layer by the dispensing technique, the method further comprises:
heating the substrate at a first heating temperature until the PDMS thin-film layer solidifies, wherein the first heating temperature ranges from 60° C. to 80° C.

5. The method of claim 1, wherein the conductive layer includes a silver paste layer; and the forming a conductive layer on the first surface by the microelectronic printing technique includes:
printing the conductive blocks through a dispensing technique using a conductive silver paste as a raw material.

6. The method of claim 5, wherein the printing the conductive blocks through a dispensing technique includes:
printing the conductive blocks through the dispensing technique based on a second printing speed and a second printing pressure, wherein the second printing speed ranges from 4 mm/s to 15 mm/s, and the second printing pressure ranges from 70 kPa to 100 kPa.

7. The method of claim 6, wherein after printing the conductive blocks through the dispensing technique, the method further comprises:
heating the substrate at a second heating temperature for a heating duration until the conductive blocks solidify, wherein the second heating temperature ranges from 80° C. to 120° C., and the heating duration ranges from 30 min to 50 min.

8. The method of claim 1, wherein the sensitive layer includes a nano-gold layer; and the forming a sensitive layer on a side of the flexible layer away from the substrate includes:
forming the thin-film blocks on the side of the flexible layer away from the substrate by a magnetron sputtering technique.

9. The method of claim 8, wherein forming the thin-film blocks on the side of the flexible layer away from the substrate by a magnetron sputtering technique includes:
sputtering the thin-film blocks on the side of the flexible layer away from the substrate for a first sputtering duration, wherein the first sputtering duration ranges from 1 min to 2 min.

10. The method of claim 9, further comprising:
modifying an antibody on a side of each of the thin-film blocks away from the flexible layer, wherein the antibody is configured to bind specifically with a to-be-tested biomolecule.

11. A biochemical sensing array, which is prepared by the method of claim 1.

12. The biochemical sensing array of claim 11, wherein the first surface of the substrate is further provided with a signal interface including a first signal interface and a plurality of second signal interfaces arranged sequentially, wherein the first signal interface is electrically connected to the first trace, and each of the plurality of second signal interfaces is electrically connected one of the second traces.

13. The biochemical sensing array of claim 12, wherein
a diameter of each of the through-holes ranges from 2.0 mm to 5.0 mm;
a distance between centers of adjacent through-holes ranges from 3.0 mm to 15 mm;
a distance between an edge of the first contact point and an edge of a corresponding through-hole ranges from 0.3 mm to 1.0 mm;
a distance between an edge of each of the second contact points and an edge of a corresponding through-hole ranges from 0.3 mm to 1.0 mm;
a line width of the first trace ranges from 0.2 mm to 0.5 mm;
a line width of each of the second traces ranges from 0.2 mm to 0.5 mm;
a line spacing between the first trace and a second trace adjacent to the first trace ranges from 0.2 mm to 0.5 mm, and a line spacing between adjacent second traces ranges from 0.2 mm to 0.5 mm;
a width of the first signal interface ranges from 0.3 mm to 0.7 mm; and
a width of each of the second signal interfaces ranges from 0.3 mm to 0.7 mm.

14. A test strip, comprising the biochemical sensing array of claim 11.

15. A test kit, comprising the test strip of claim 14.

16. The method of claim 1, wherein
the biochemical sensing array includes a plurality of biochemical sensors corresponding to the through-holes, and at least two of the biochemical sensors are different;
the sensitive layer further includes a microfluidic channel and a target sample region, and the microfluidic channel is in communication with the target sample region and the plurality of biochemical sensors;
the method further includes:
determining a microfluidic channel feature based on antibody information of the plurality of biochemical sensors and dimensional information of the through-holes; and
fabricating the microfluidic channel using the microelectronic printing technique based on the microfluidic channel feature.

17. The method of claim 16, wherein the microfluidic channel includes a plurality of sub-channels that are in communication with a plurality of biochemical sensors;
a size-selective micropillar array is provided upstream of the sub-channels, and a dimension of the size-selective micropillar array is related to the antibody information of the biochemical sensors connected to the microfluidic channel;
a spiral microchannel is provided downstream of the sub-channels.

18. The method of claim 16, wherein the microfluidic channel includes one or more microvalves configured to adjust a flow direction of the microfluidic channel.

19. The method of claim 18, wherein the method further includes:
during a trial manufacturing stage of the biochemical sensing array, determining a target opening/closing state of each of the one or more microvalves based on an operating mode and a real-time resistance value of the biochemical sensing array.

20. The method of claim 16, wherein the method further includes:
determining a target magnetron sputtering parameter based on the microfluidic channel feature and a preset magnetron sputtering parameter, wherein the preset magnetron sputtering parameter corresponds to the antibody information of the biochemical sensors and the dimensional information of the through-holes; and
preparing the nano-gold layer using a magnetron sputtering instrument based on the target magnetron sputtering parameter.
